(19) United States
(12) Patent Application Publication
(10) Pub. No.: US 2023/0343444 A1
(43) Pub. Date: Oct. 26, 2023

(54) SMART INVENTORY MANAGEMENT CABINET

(71) Applicant: Johnson & Johnson Surgical Vision, Inc., Irvine, CA (US)

(72) Inventors: Edward R Kernick, Jacksonville, FL (US); Yasemar R. Perez, Jacksonville, FL (US)

(21) Appl. No.: 17/965,555

(22) Filed: Oct. 13, 2022

Related U.S. Application Data
(60) Provisional application No. 63/334,421, filed on Apr. 25, 2022.

(51) Int. Cl. G16H 40/20; A47F 3/02; A47F 7/02

(52) U.S. Cl. CPC G16H 40/20; A47F 3/02; A47F 7/02

(57) ABSTRACT

Inventory management cabinets, systems, and related methods are described herein. An example inventory management cabinet includes a housing defining a storage area, the storage area being configured to receive a product, and a plurality of slots arranged within the housing, each of the slots being configured to receive a respective unit of the product. The cabinet also includes at least one imaging device configured to capture information about the product. The cabinet further includes a controller operably coupled to the at least imaging device, where the controller includes a processor and a memory having computer-executable instructions stored thereon. The controller is configured to detect activity within the storage area of the housing, and control the at least one imaging device to initiate capture one or more images of the product in response to detecting activity within the storage area of the housing.

### CROSS-REFERENCE TO RELATED APPLICATIONS

This application claims the benefit of U.S. provisional patent application No. 63/334,421, filed on Apr. 25, 2022, and titled “SMART INVENTORY MANAGEMENT CABINET,” the disclosure of which is expressly incorporated herein by reference in its entirety.

### BACKGROUND

The present disclosure relates to smart inventory management cabinets for surgical implants. For example, the smart inventory management cabinets may be designed to store ophthalmic lenses, and more particularly to systems to dispense ophthalmic objects, record and track patient information, determine different lenses for patients, and to track and control inventory of ophthalmic lenses in the offices of eye care professionals.

### SUMMARY

Inventory management cabinets, systems, and related methods are described herein.

An example inventory management cabinet includes a housing defining a storage area, the storage area being configured to receive a product, and a plurality of slots arranged within the housing, each of the slots being configured to receive a respective unit of the product. The cabinet also includes at least one imaging device configured to capture information about the product. The cabinet further includes a controller operably coupled to the at least imaging device, where the controller includes a processor and a memory having computer-executable instructions stored thereon. The controller is configured to detect activity within the storage area of the housing, and control the at least one imaging device to initiate capture one or more images of the product in response to detecting activity within the storage area of the housing.

Additionally, activity within the storage area of the housing is optionally detected using the at least one imaging device.

Alternatively or additionally, the cabinet includes a motion sensor configured to detect presence of an object within the storage area of the housing. Optionally, activity within the storage area of the housing is detected using the motion sensor.

Alternatively or additionally, the cabinet includes a plurality of presence sensors configured to detect presence of a respective unit of the product in a respective one of the plurality of slots. Optionally, activity within the storage area of the housing is detected using the presence sensors. Alternatively or additionally, each of the presence sensors includes a light emitter and a photodetector.

Alternatively or additionally, the controller is further configured to extract information from the one or more images of the product captured by the at least one imaging device. The extracted information is used to inventory the product. For example, the controller is further configured to extract information from the one or more images of the product captured by the at least one imaging device by receiving the one or more images of the product from the at least one imaging device, and analyzing the one or images of the product to extract respective product identifiers associated with respective units of the product. The controller is further configured to inventory the product based, at least in part, on the extracted information, for example, by decoding the respective product identifiers associated with the respective units of the product, and using the respective product identifiers, associating the respective units of the product with the respective slots. Optionally, each of the respective product identifiers is a one-dimensional (1D) barcode, a two-dimensional (2D) barcode, a three-dimensional (3D) barcode, a universal product code (UPC), a stock keeping unit (SKU), text, or a graphic.

Alternatively or additionally, the cabinet includes a plurality of visual indicators configured to indicate respective positions of the respective units of the product within the housing. Optionally, each of the visual indicators is at least one of a light emitter or a graphical display. In some implementations, the housing includes an external frame, and at least one of the visual indicators is arranged on or adjacent to the external frame. Alternatively or additionally, in some implementations, a respective visual indicator is arranged on, within, or adjacent to each one of the respective slots.

Alternatively or additionally, the cabinet includes a plurality of imaging devices, each of the imaging devices being configured to capture information about the product located in a respective region of the storage area of the housing.

Alternatively or additionally, the cabinet includes a human machine interface (HMI) configured to provide a communication interface between a user and the inventory management cabinet.

Alternatively or additionally, each of the respective units of the product is a product package. Optionally, the product package includes a surgical implant, for example an intraocular lens or an orthopedic implant. Optionally, the product package includes a surgical tool.

Alternatively or additionally, the controller is further configured to transmit an inventory of the product over a network to a remote system such as a remote database, for example.

Alternatively or additionally, the controller is further configured to receive a request for the desired unit of the product, transmit the request for the desired unit of the product over a network to a remote system, and receive a response from the remote system. The response includes a slot where the desired unit of product is located. Optionally, the remote system comprises a database.

Another example inventory management cabinet includes a housing defining a storage area, the storage area being configured to receive a product, and a plurality of drawers arranged within the housing, each of the drawers comprising a plurality of slots, each of the slots being configured to receive a respective unit of the product. The cabinet also includes a plurality of imaging devices configured to capture information about the product. The cabinet further includes a controller operably coupled to the at least imaging device, where the controller includes a processor and a memory having computer-executable instructions stored thereon. The controller is configured to detect activity within the storage area of the housing, and control the imaging devices to initiate capture one or more images of the product in response to detecting activity within the storage area of the housing.

An example modular inventory management system is also described herein. The system includes a first inventory management cabinet and a second inventory management cabinet. Additionally, the system includes a human machine interface (HMI) configured to provide a communication interface between a user and the first and second inventory management cabinets.

An example automated method for inventory management is also described herein. The method includes detecting activity within a storage area of a housing of an inventory management cabinet; automatically initiating capture one or more images of a product within the storage area of the housing in response to detecting activity within the storage area of the housing; and inventorying the product based, at least in part, using information extracted from the one or more images of the product. The method optionally further includes providing the inventory management cabinet. This disclosure contemplates that the inventory management cabinet is a cabinet as described above.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a perspective view of an example inventory management cabinet.

FIG. 2 is another perspective view of the example inventory management cabinet of FIG. 1.

FIG. 3 is a cross section view of the example inventory management cabinet of FIG. 1 showing a plurality of imaging devices.

FIG. 4 is a zoomed in view of the region shown in the dashed box of FIG. 3.

FIG. 5 is a perspective view of a portion of the example inventory management cabinet of FIG. 1 showing a plurality of imaging devices.

FIG. 6 is a perspective view of a portion of the example inventory management cabinet of FIG. 1 showing a motion sensor.

FIG. 7 is a side view of a portion of the example inventory management cabinet of FIG. 1 showing a plurality of presence sensors.

FIG. 8 is a zoomed-in view of a portion of FIG. 7.

FIG. 9 is a perspective view of a portion of the example inventory management cabinet of FIG. 1 showing a plurality of slots in the storage area.

FIG. 10 is a perspective view of a portion of the example inventory management cabinet of FIG. 1 showing a plurality of visual indicators.

FIG. 11 is a perspective view of a portion of the example inventory management cabinet of FIG. 1 showing the electronics compartment including the controller.

FIG. 12 is a perspective view of a portion of the example inventory management cabinet of FIG. 1 showing a cut-away portion in the housing that allows a user to access the cabinet and an output device (e.g., speakers).

FIG. 13 is a system overview of the inventory management cabinet in an example described herein.

FIG. 14 is a schematic diagram of system inputs for the inventory management cabinet in an example described herein.

FIG. 15 is a schematic diagram of system outputs for the inventory management cabinet in an example described herein.

FIG. 16 is a diagram showing a plurality of product slots (by row and column) for the inventory management cabinet in an example described herein.

FIG. 17 is a flow diagram illustrating example process operations for the inventory management cabinet in an example described herein.

FIG. 18 is an architecture diagram for the inventory management cabinet in an example described herein.

FIG. 19 is a perspective view of a modular system including a plurality of inventory management cabinets.

FIG. 20 is an example computing device.

FIG. 21 is an example operating environment for the implementations described herein.

FIGS. 22A-22B are perspective views of another example inventory management cabinet. FIG. 22A illustrates a four-drawer implementation with dummy storage space. FIG. 22B illustrates a four-drawer implementation without dummy storage space.

FIG. 23 is a perspective view of the cabinet shown in FIG. 22B with an open drawer.

FIGS. 24A-24C illustrate components within the drawers of the cabinets shown in FIGS. 22A-23. FIG. 24A illustrates a tray. FIG. 24B illustrates dividers, which can be arranged in the tray.

FIG. 24C illustrates a plurality of trays arranged inside a drawer with and without units of product.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. As used herein, the terms “about” or “approximately” when referring to a measurable value such as an amount, a percentage, and the like, is meant to encompass variations of ±20%, 10%, 5%, or +1% from the measurable value.

Example Cabinets, Systems, and Related Methods

Inventory management cabinets, systems, and methods are described herein. Such cabinets, systems, and methods can be used to track/inventory product such as contact lenses. For example, the cabinets, systems, and methods described herein are capable of: (i) keeping track of units of product removed from storage, (ii) informing the user of stocking needs, (iii) automatically placing orders for product, (iv) including storage space for all regularly prescribed lenses, and/or (v) working during power outages.

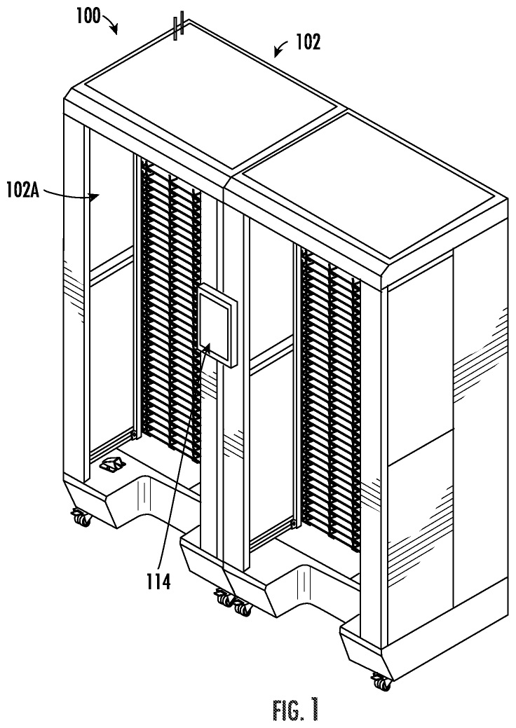

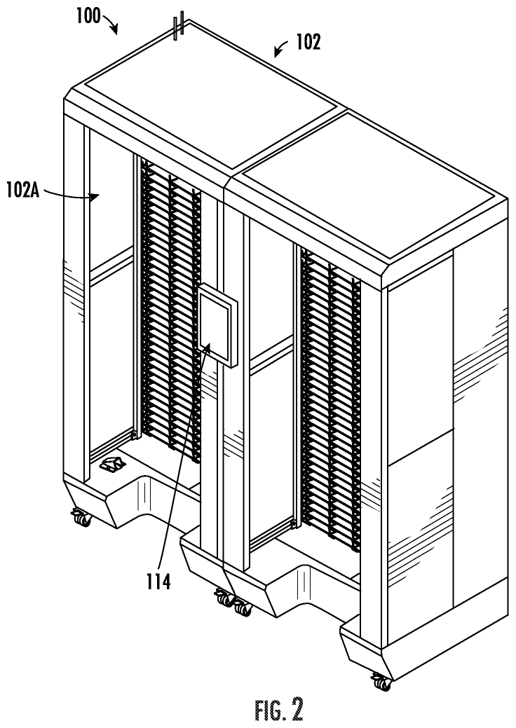

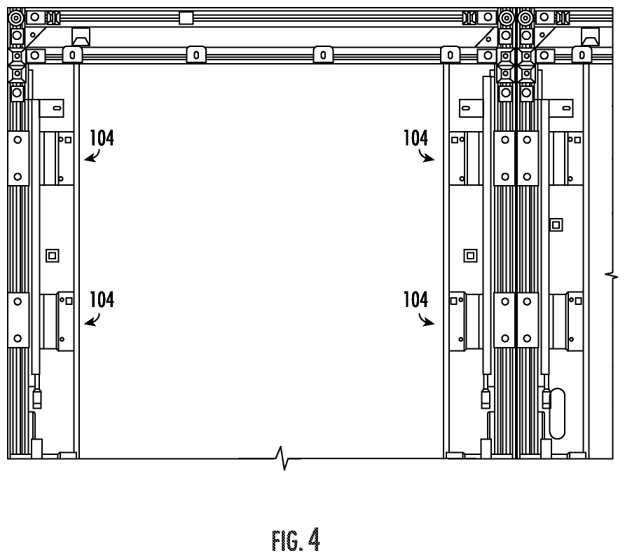

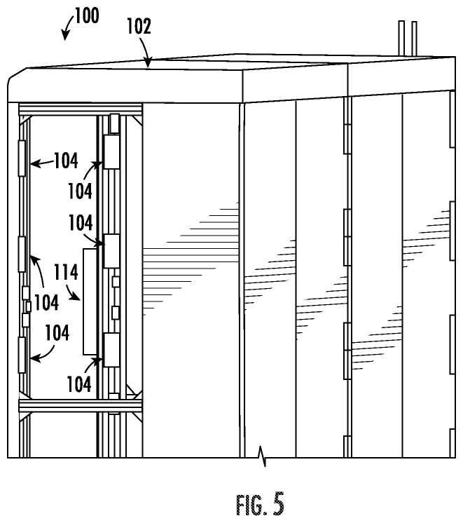

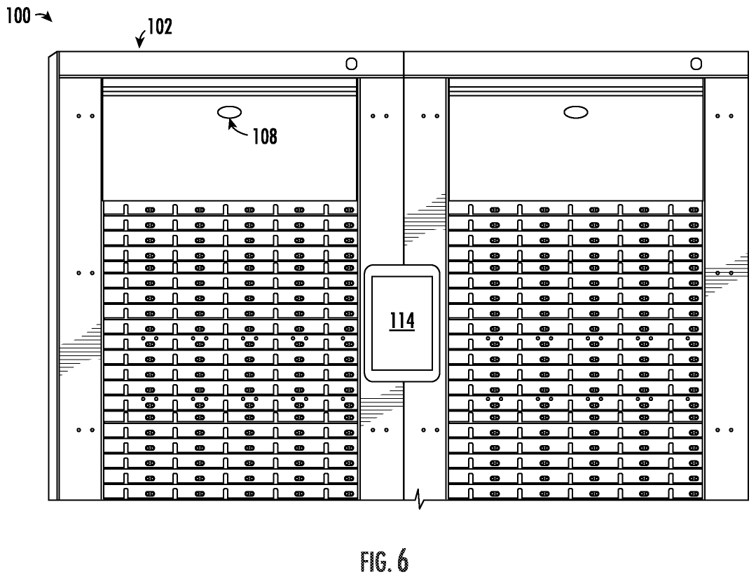

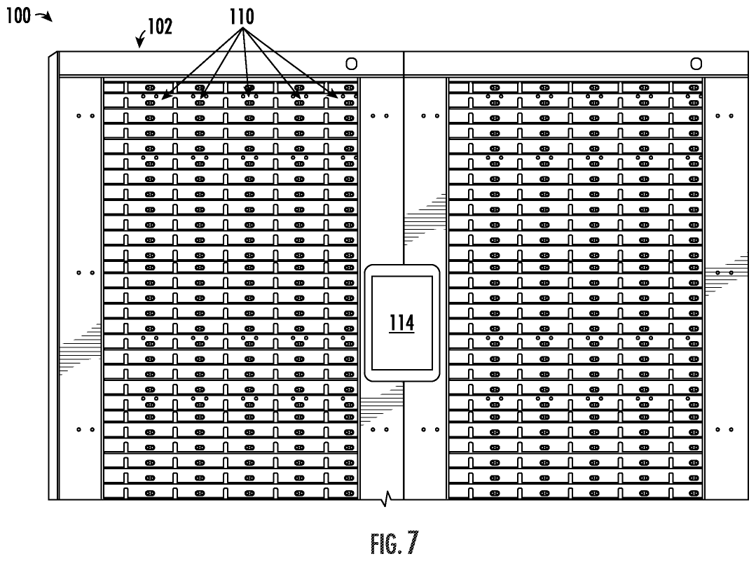

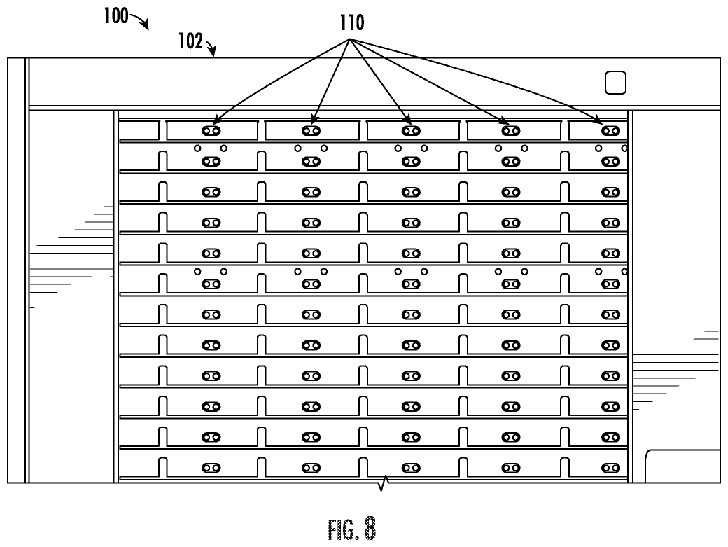

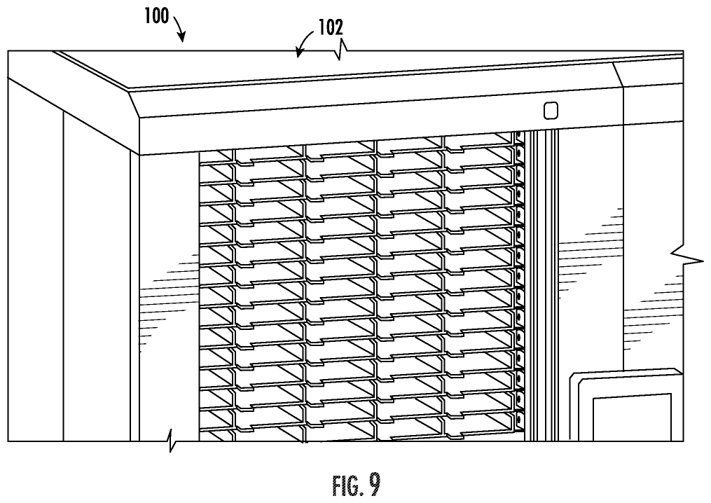

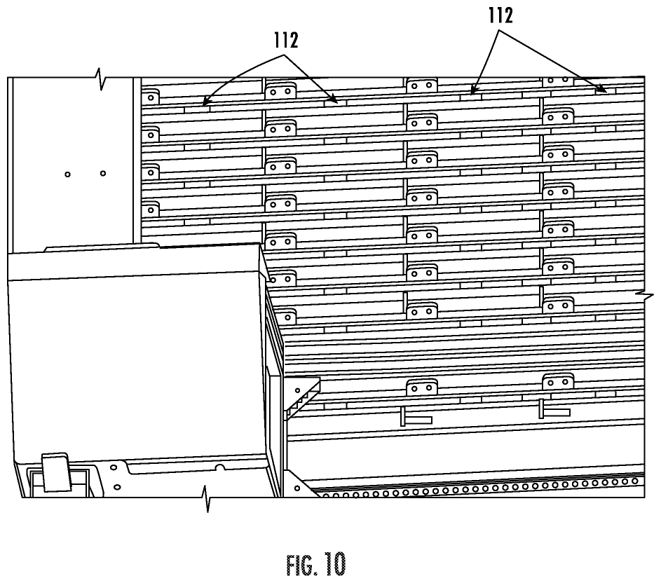

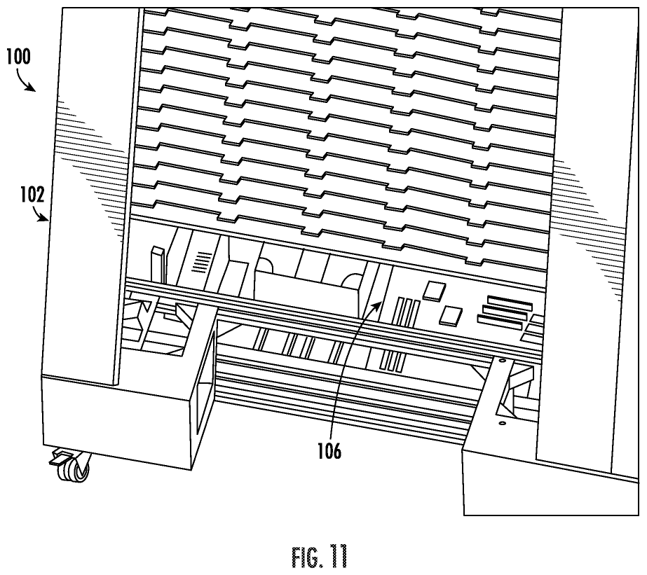

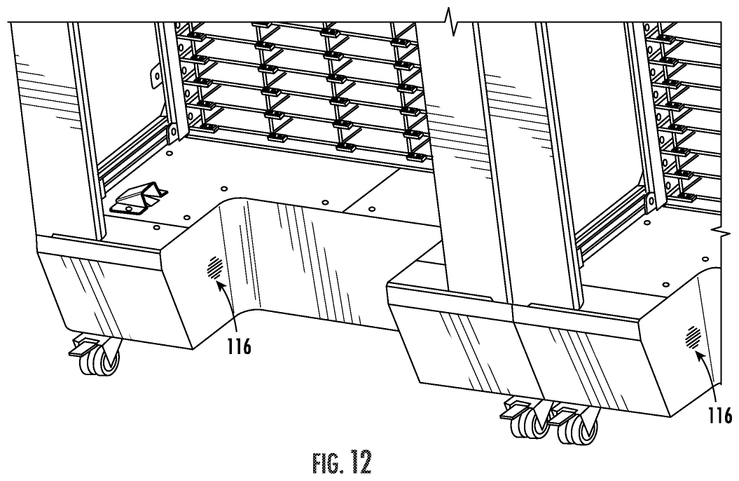

Referring now to FIGS. 1-12, an inventory management cabinet 100 is shown. Inventory management operations are described in US 2020-0364648 and US 2020-0364650, the disclosures of which are incorporated herein by reference in their entireties. Such operations may include inventorying product and facilitating product retrieval requests.

The cabinet 100 includes a housing 102 defining a storage area 102a, the storage area 102a being configured to receive a product, and a plurality of slots arranged within the housing 102, each of the slots being configured to receive a respective unit of the product. Each of the respective units of the product is a product package. Optionally, the product package includes a surgical implant, for example an intraocular lens. It should be understood that an intraocular lens is provided only as an example. This disclosure contemplates that the product package may include other items or objects including, but not limited to, orthopedic implants (e.g., patella, tibial, femoral, spinal, etc. components) or surgical tools.

The cabinet 100 also includes at least one imaging device 104 configured to capture information about the product. Optionally, the cabinet includes a plurality of imaging devices, each of the imaging devices being configured to capture information about the product located in a respective region of the storage area 102a of the housing 102. As described herein, the imaging devices can optionally be arranged throughout the storage area 102a, each imaging device being configured to capture images of different regions of the storage area 102a. The imaging device 104 is coupled to a controller (discussed below) through one or more communication links. This disclosure contemplates the communication links are any suitable communication link. For example, a communication link may be implemented by any medium that facilitates data exchange including, but not limited to, wired, wireless and optical links. This allows the imaging device 104 and the controller (discussed below) to exchange data (e.g., image data, control signals). Optionally, the imaging device 104 is a digital camera such as a universal serial bus (USB) digital camera. The digital camera may capture compressed images (e.g., MJPEG format) or uncompressed images (e.g., UYVY format). One example suitable USB digital camera is See3CAM_130-13MP 4K or more generally e-con Systems See3Cam series. Another example of myriad suitable cameras would be the USB 3.1 High Resolution Autofocus Camera. Imaging devices are known in the art and therefore not described in further detail herein.

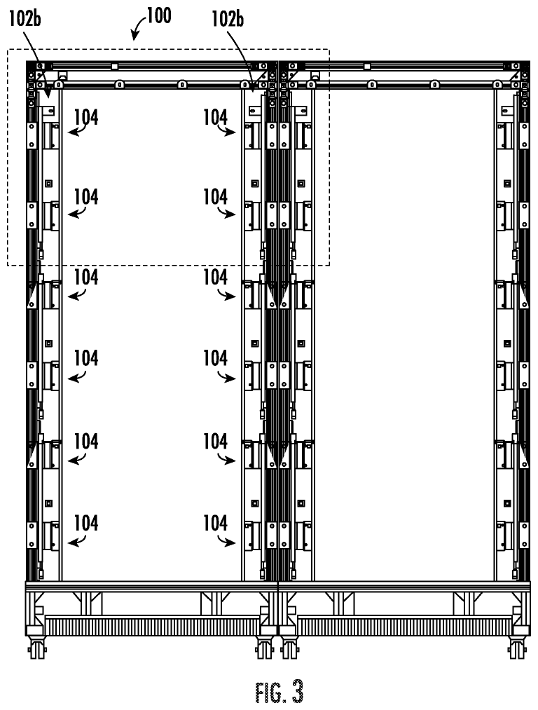

As described above, the cabinet 100 can include a plurality of imaging devices 104, and the imaging devices 104 can be arranged throughout the storage area 102a. For example, in FIG. 3, there are twelve imaging devices 104, where 6 imaging devices 104 are arranged on each of right and left vertical supports 102b of the housing 102. The vertical supports 102b are on the front side of the housing 102, and the imaging devices 104 are mounted such that the product within the storage area 102a is within the fields of view of the imaging devices 104. In FIG. 3, the imaging devices 104 are arranged with equal spacing from one another along the vertical supports 102b. Each of the imaging devices 104 is configured to capture images of different regions of the storage area 102a. Such regions may or may not overlap with regions captured by other imaging devices 104. It should be understood that the number and/or arrangement of imaging devices 104 shown in FIG. 3 are provided only as examples. This disclosure contemplates providing a cabinet with a different number and/or arrangement of imaging devices.

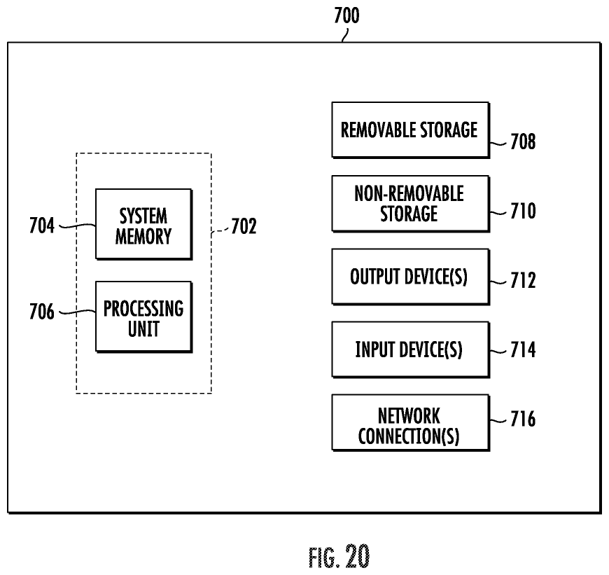

In one implementation, the cabinet can include twelve digital cameras, for example USB cameras, and the digital cameras can be mounted in the cabinet as described above with regard to FIG. 3. Each of the USB cameras is coupled to a controller through a USB cable (e.g., a communication link) via a peripheral component interconnect (PCI) card. Each PCI includes a plurality of USB ports (e.g., up to five USB ports). Accordingly, the USB cameras can be coupled to the controller via multiple PCI cards. This disclosure contemplates connecting one, two, three, four, or five USB cameras to a single PCI card. In one aspect, the controller to which the USB cameras are connected is a central processing unit (CPU). A CPU includes at least one or more processing units and memory (see e.g., FIG. 20). CPUs are well known in the art and therefore not described further herein. In this aspect, the cabinet performs a full scan with all USB cameras in less than about 2 seconds, where the full scan includes a computer vision-based analysis for presence/absence of a unit or units of product in the cabinet. In another aspect, the controller to which the USB cameras are connected is a graphics processing unit (GPU). A GPU is specialized device for handing images and graphics and includes at least one or more processing units and memory (see e.g., FIG. 20). GPUs are well known in the art and therefore not described further herein. In this aspect, the cabinet performs a full scan with all USB cameras in less than 1 second, where the full scan includes a computer vision-based analysis for presence/absence of a unit or units of product in the cabinet. The GPU runs frames in a rate adequate for the application suitable for the desired user experience of the system and in this particular non-limiting example at about 50 frames per second. In either aspect, the USB cameras simultaneously capture images and these images are processed by the controller. It should be understood that the number of USB cameras provided above is only an example. This disclosure contemplates providing a cabinet with more than 12 USB cameras (e.g., 13, 14, 15, . . . 24 or more USB cameras) or less than 12 USB cameras (e.g., 11, 10, 9, . . . 1 USB cameras).

The cabinet 100 further includes a controller 106 operably coupled to the at least imaging device 104, where the controller 106 includes a processor and a memory having computer-executable instructions stored thereon. This disclosure contemplates that the HMI 114 includes a computing device (e.g., computing device 700 of FIG. 20). In some implementations, the controller 106 can be implemented using a CPU or a GPU. In some implementations, the controller 106 can be implemented using a single board computer (SBC), one or more microcontrollers (MCU), or combinations thereof. One suitable SBC is the AAEON MAX-C246 Motherboard SBC from Aaeon Technology Inc. of New Taipei City, Taiwan. One suitable MCU is an SPC560P50L5 Series 32-bit Microcontroller from STMicroelectronics of Geneva, Switzerland. The controller 106 is configured to detect activity within the storage area 102a of the housing 102, and control the at least one imaging device 104 to initiate capture one or more images of the product in response to detecting activity within the storage area 102a of the housing 102. As used herein, the activity detected may be an object (e.g., a user's hand and/or arm or any other part of a user's body or extension thereof) in the storage area 102a and/or a change of state (e.g., addition or removal of product) with the storage area 102a.

In some implementations described herein, the cabinet 100 uses computer vision to detect activity within the storage area 102a of the housing 102. In these implementations, the imaging device or devices continuously capture images of the product within the storage area 102b and such images are processed with computer vision methods to detect changes (e.g., restocking or removal of product). Optionally, in some implementations, the image capture and computer vision methods are triggered in response to activity detection by motion and/or the presence sensors described below.

Activity within the storage area 102a of the housing 102 is optionally detected using the at least one imaging device 104. For example, the controller 106 can be configured to receive one or more images captured by the at least one imaging device 104 and analyze the received images to identify an object such as a user's body part and/or product addition/removal. This disclosure contemplates using any known techniques to detect activity from images including, but not limited to, computer vision. Optionally, activity detection is performed in real time.

Alternatively or additionally, the cabinet 100 optionally includes a motion sensor 108 configured to detect presence of an object within the storage area 102a of the housing 102. Optionally, an output of the motion sensor 108 can be used to trigger the computer vision methods described below. The motion sensor 108 is coupled to the controller 106 through one or more communication links. This disclosure contemplates the communication links are any suitable communication link. For example, a communication link may be implemented by any medium that facilitates data exchange including, but not limited to, wired, wireless and optical links. This allows the controller 106 and the motion sensor 108 to exchange data (e.g., sensed signals, control signals, etc.). Optionally, activity within the storage area 102a of the housing 102 is detected using the motion sensor 108. Example motion sensors include passive infrared (IR) or microwave sensors. Passive IR sensors sense changes in temperature between background and objects (e.g., human body parts). Microwave sensors continuously emit radio waves and detect frequency shift in the reflected waves, which are the result of moving objects. Motion sensors are known in the art and therefore not described in further detail herein. This disclosure contemplates that the motion sensor 108 can be arranged on, within, or adjacent to an external frame of the housing 102. Optionally, the motion sensor 108 is arranged within the storage area 102a. It should be understood that the cabinet may include more than one motion sensor, i.e., a plurality of motion sensors. Optionally, activity detection is performed in real time.

Alternatively or additionally, the cabinet 100 optionally includes a plurality of presence sensors 110 configured to detect presence of a respective unit of the product in a respective one of the plurality of slots. Optionally, an output of the presence sensors 110 can be used to trigger the computer vision methods described below. The presence sensors 110 are coupled to the controller 106 through one or more communication links. This disclosure contemplates the communication links are any suitable communication link. For example, a communication link may be implemented by any medium that facilitates data exchange including, but not limited to, wired, wireless and optical links. This allows the controller 106 and the presence sensors 110 to exchange data (e.g., sensed signals, control signals, etc.). Optionally, activity within the storage area 102a of the housing 102 is detected using the presence sensors 110. This disclosure contemplates a presence sensor can be arranged on, within, or adjacent to a slot. For example, a presence sensor can be a light emitter and a photodetector. Example presence sensors 110 include IR sensors, which include an IR emitter and an IR photodetector. In such a sensor, the IR photodetector's electrical properties are variable depending on the intensity of IR light. Accordingly, the presence or absence of a unit of the product can be detected by a change in the IR detector's output voltage. For example, when the unit of product is removed, the IR photodetector senses the IR beam, but when the unit of product is present, the IR beam is interrupted, each state being associated with a different detector output voltage. Optionally, activity detection is performed in real time.

The controller 106 is further configured to extract information from the one or more images of the product captured by the at least one imaging device 104. The extracted information is used to inventory the product. These operations can be initiated in response to detecting activity within the storage area 102a, which can be accomplished using computer vision, motion sensors, presence sensors, or combinations thereof. For example, the controller 106 can be configured to receive images of the product captured by the imaging device 104, analyze the images of the product to extract respective product identifiers associated with respective units of product, and inventory the product, based at least in part, on the extracted information. This disclosure contemplates using any known techniques to analyze the images of the product including, but not limited to, computer vision. Optionally, image analysis is performed in real time. Optionally, the step of inventorying the product based, at least in part, on the extracted information can include decoding the respective product identifiers associated with the respective units of the product, and using the respective product identifiers, associating the respective units of the product with the respective slots. Each of the respective product identifiers is a one-dimensional (1D) barcode, a two-dimensional (2D) barcode, a three-dimensional (3D) barcode, a universal product code (UPC), a stock keeping unit (SKU), text, or a graphic.

Alternatively or additionally, the cabinet 100 optionally includes a plurality of visual indicators 112 configured to indicate respective positions of the respective units of the product within the housing 102. The visual indicators 112 are coupled to the controller 106 through one or more communication links. This disclosure contemplates the communication links are any suitable communication link. For example, a communication link may be implemented by any medium that facilitates data exchange including, but not limited to, wired, wireless and optical links. This allows controller 106 and the visual indicators 112 to exchange data (e.g., control signals, etc.). Each of the visual indicators 112 can be a light emitter (e.g., LED) or a graphical display. Additionally, each of the visual indicators 112 can be arranged on, within, or adjacent to each one of the respective slots. Visual indicators 112 can be used to identify product, for example responsive to a user request, by illumination. Such illumination can be altered by intensity, flashing ON/OFF, etc. Additionally, visual indicators 112 can be used to identify product by expiration date (e.g., the product closest to expiration is identified by flashing light).

Alternatively or additionally, the cabinet 100 includes a human machine interface (HMI) 114 configured to provide a communication interface between a user and the inventory management cabinet. As described herein, the HMI 114 is coupled to the controller 106 and/or a remote server through one or more communication links. This disclosure contemplates the communication links are any suitable communication link. For example, a communication link may be implemented by any medium that facilitates data exchange including, but not limited to, wired, wireless and optical links. This allows the HMI 114 and the controller 106 and/or remote server to exchange data (e.g., sensed data, control signals, etc.). This disclosure contemplates that the HMI 114 includes a computing device (e.g., computing device 700 of FIG. 20). In some implementations, the HMI 114 is a tablet computer. Alternatively, in other implementations, the HMI 114 is a laptop computer, desktop computer, or mobile computing device.

Alternatively or additionally, the cabinet 100 includes an output device such as a speaker 116. The output device is coupled to the controller 106 through one or more communication links. This disclosure contemplates the communication links are any suitable communication link. For example, a communication link may be implemented by any medium that facilitates data exchange including, but not limited to, wired, wireless and optical links. This allows the output device and the controller 106 to exchange data (e.g., control signals, etc.). The controller 106 is further configured to generate an alarm using the output device. Such alarm may be audible, visual, or combinations thereof.

Alternatively or additionally, the cabinet includes a power supply, which is optionally arranged in the housing 102. This disclosure contemplates that the power supply can be used to provide power to one or more of the electrical components described herein. The power supply can optionally be a battery. For example, the cabinet can be configured to connect to grid power (e.g., standard alternating current (A/C) power delivered to homes/businesses) during normal operation. The power supply can deliver electrical power to the cabinet in response to disruption (e.g., power outages).

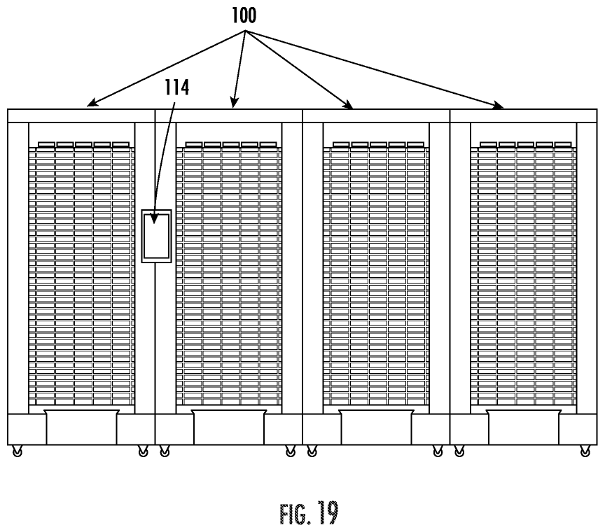

A modular inventory management system is also described herein. The system includes a first inventory management cabinet and a second inventory management cabinet. This disclosure contemplates that the first and second inventory management cabinets are cabinets as described above. Additionally, the system includes a human machine interface (HMI) configured to provide a communication interface between a user and the first and second inventory management cabinets. A modular system is shown in FIG. 19.

An automated method for inventory management is also described herein. The method includes detecting activity within a storage area of a housing of an inventory management cabinet; automatically initiating capture one or more images of a product within the storage area of the housing in response to detecting activity within the storage area of the housing; and inventorying the product based, at least in part, using information extracted from the one or more images of the product. The method optionally further includes providing the inventory management cabinet. This disclosure contemplates that the inventory management cabinet is a cabinet as described above.

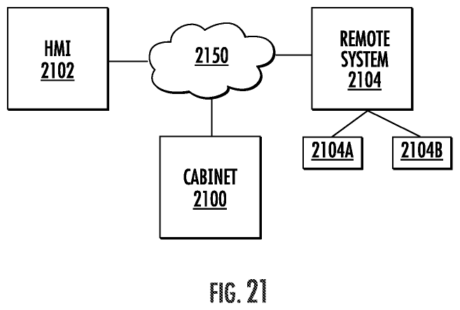

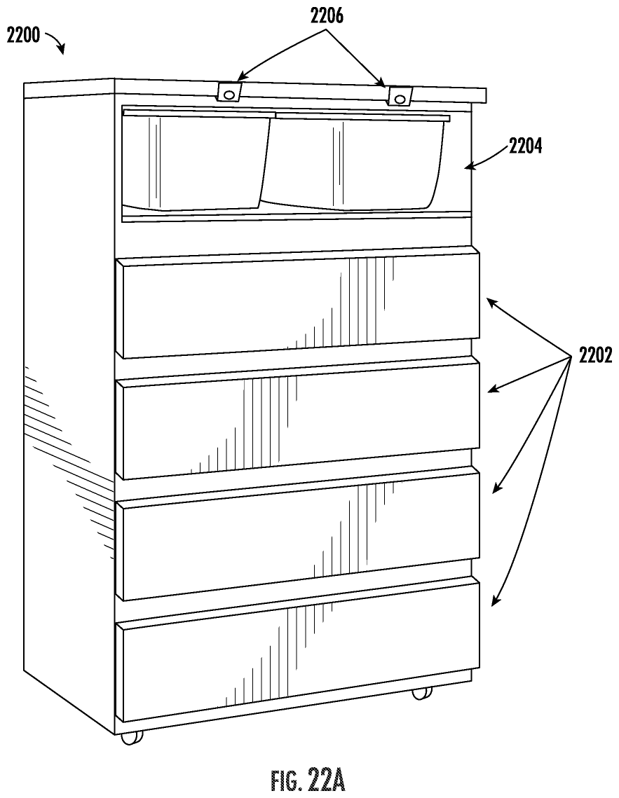

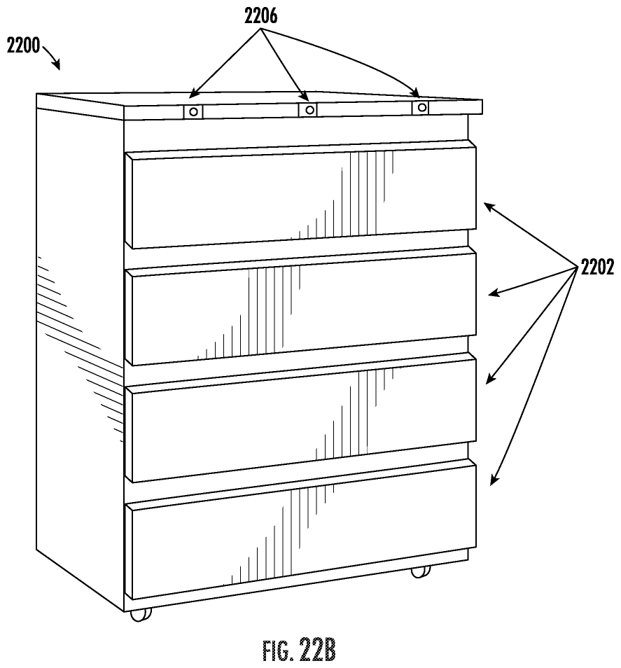

Referring now to FIG. 21, an example operating environment for the implementations described herein is shown. As shown in FIG. 21, an inventory management cabinet 2100, a human machine interface 2102, and a remote system 2104 can be operably coupled by one or more networks 2150. The inventory management cabinet 2100 is described in detail herein. For example, the inventory management cabinet 2100 may be the cabinet 100 described with respect to FIGS. 1-12 above or the cabinet 2200 described with respect to FIGS. 22A-24C below. Additionally, the human machine interface 2102 may be the HMI 114 described with respect to FIGS. 1-12 above. The remote system 2104 can be a computing device (e.g., computing device 700 of FIG. 20) such as a server. Optionally, in some implementations, the remote system 2104 is a cloud-based system, e.g., one or more computer system resources such as processors and data storage devices that are allocated to serve the needs of the human machine interface 2102 on demand. Cloud-based systems are known in the art and not described in further detail below. In some implementations, the remote system 2104 can include or access a database 2104A. Alternatively or additionally, the remote system 2104 can include or access electronic medical records (EMRs) 2104B.

As discussed above, the inventory management cabinet 2100, human machine interface 2102, and remote system 2104 discussed above can be connected by one or more networks 2150. This disclosure contemplates that the networks 2150 are any suitable communication network. The networks can be similar to each other in one or more respects. Alternatively or additionally, the networks can be different from each other in one or more respects. The networks 2150 can include a local area network (LAN), a wireless local area network (WLAN), a wide area network (WAN), a metropolitan area network (MAN), a virtual private network (VPN), etc., including portions or combinations of any of the above networks. The inventory management cabinet 2100, human machine interface 2102, and remote system 2104 can be coupled to the networks 2150 through one or more communication links. This disclosure contemplates the communication links are any suitable communication link. For example, a communication link may be implemented by any medium that facilitates data exchange including, but not limited to, wired, wireless and optical links. Example communication links include, but are not limited to, a LAN, a WAN, a MAN, Ethernet, the Internet, or any other wired or wireless link such as WiFi, WiMax, 3G, 4G, or 5G.

This disclosure contemplates that the inventory management cabinet 2100, human machine interface 2102, and remote system 2104 can interact to carry out the inventory and shipment/distribution functionalities as described in U.S. 2019-0311316, the disclosure of which is expressly incorporated herein by reference in its entirety. For example, as described below, the remote system 2104 can manage/maintain a database 2104A reflecting the inventory of product (e.g., contact lenses) stored in the inventory management cabinet 2100. By exchanging messages over the networks 2150, the remote system 2104 can receive messages with product inventory updates from the inventory management cabinet 2100. The remote system 2104 can also query the database 2104A in response to requests from the inventory management cabinet 2100 and/or the human machine interface 2102. This disclosure contemplates that a user (e.g., a healthcare professional such as an eye care professional (ECP)) can interact with the inventory management cabinet 2100 and/or the remote system 2104 using the human machine interface 2102. For example, the human machine interface 2102 can run an application and/or interface with the inventory management cabinet 2100 and/or the remote system 2104 using a web browser.

As described herein, a controller of the inventory management cabinet (e.g., controller 106 of FIGS. 1-12) can be configured to inventory the product based, at least in part, on the information about the product, and cause one or more of the visual indicators (e.g., visual indicators 112 of FIGS. 1-12) that are associated with a desired unit of the product to actuate. An example process is now described. First, a user (e.g., ECP) enters a request for a desired unit of product using the human machine interface 2102. The human machine interface 2102 can transmit the request for the desired unit of the product to the inventory management cabinet 2100 via a network. A controller of the inventory management cabinet 2100 can be configured to receive a request for the desired unit of the product. Additionally, the controller can be further configured to transmit the request for the desired unit of the product over the network to the remote system 2104. As described herein, the remote system 2104 can include and/or access an inventory database such as database 2104A. The remote system can query the database to determine the position(s) of the desired unit(s) of product within the inventory management cabinet 2100. The remote system can transmit a response to the controller over the network, and the controller can receive the response, which includes position(s) of the desired unit(s) of product within the storage area. It should be understood that such position(s) can include specific slots in the storage area of the cabinet. As described herein, the controller can be configured to transmit signals to actuate visual indicators to assist the user in identifying the position(s) of the desired unit(s) of product. These visual indicators highlight the locations of the desired units of the product for the benefit of the user.

The user then removes the desired units of the product. As described herein, activity within the storage area of the cabinet can be detected by the inventory management cabinet 2100, e.g., using the imaging devices, motion sensors, and/or presence sensors. This causes the controller to initiate a computer vision process. By initiating the computer vision process, the inventory management cabinet 2100 can read/decode the machine-readable labels (e.g., barcodes, UPC, SKU, text, graphics) associated with the units of the product. The respective units of the product can then be associated with respective positions within the storage area. The respective positions for each of the units of product can then be transmitted by the controller to the remote system. In other words, the controller can be configured to transmit the updated inventory of the product over the network to the remote system, and the database can be updated accordingly.

Alternatively or additionally, the inventory management cabinet 2100 can be restocked effortlessly. For example, the user (e.g., ECP) can restock product by placing the product packages in any empty slots in the storage area. Unlike conventional storage system, there is no need to organize the storage in any manner, for example, by prescription, power, type, etc. The product packages can instead be placed at random in the storage area. Upon detecting activity within the storage area, the controller can initiate the computer vision process. By initiating the computer vision process, the inventory management cabinet 2100 can read/decode the machine-readable labels (e.g., barcodes, UPC, SKU, text, graphics) associated with the units of the product. The respective units of the product can then be associated with respective positions within the storage area. The respective positions for each of the units of product can then be transmitted by the controller to the remote system. In other words, the controller can be configured to transmit the updated inventory of the product over the network to the remote system, and the database can be updated accordingly.

As described herein, the imaging device of the inventory management cabinet 2100 can be a digital camera, which is capable of capturing images of machine readable product identifiers such as a 1D barcode, a 2D barcode, a 3D barcode, a UPC, or an SKU. An imaging device is also capable of capturing images of text and/or a graphics, which can serve as machine readable product identifiers. For example, text and/or graphics can include, but are not limited to, brand name, product name, product description, logo, etc. In these implementations, image processing techniques can be used to decode the machine readable product identifiers. Accordingly, the step of inventorying the product based, at least in part, on the information about the product can include receiving images of the product captured by the imaging device, analyzing the images of the product to identify respective product identifiers associated with respective units of the product, decoding the respective product identifiers associated with the respective units of the product. After analyzing/decoding the respective product identifiers, it is possible to associate the respective units of the product with the respective positions within the storage area. This disclosure contemplates performing this association with either the controller and/or the remote system 2104.

Optionally, in some implementations, the step of inventorying the product based, at least in part, on the information about the product further includes cropping a portion of the images of the product. By cropping the images, it is possible to focus on the portion of the image expected to contain the product identifiers. Thus, the cropped portion of the images is analyzed to identify the respective product identifiers associated with the respective units of the product. Additionally, the controller can be configured to transmit the images of the product over a network to the remote system 2104. In these implementations, the images can be stored by the remote system for back up purposes, or image processing (some or all) can be offloaded from the controller to the remote system. Alternatively or additionally, the controller can be configured to store the images of the product in the memory. In some implementations, the images can be stored only temporarily (e.g., to allow for image processing) and then written over to minimize the storage requirements at the inventory management cabinet 2100.

Optionally, in some implementations, the step of inventorying the product based, at least in part, on the information about the product further includes analyzing the images of the product to identify one or more of the respective positions within the storage area associated with a missing, unrecognized, or unreadable product identifier. Optionally, the controller can be configured to distinguish between missing units of product and units of product having unrecognized/unreadable product identifiers. It should be understood that the former may be restocked, while the latter may be repositioned (e.g., flipped over, turned over, relabeled) to correctly orient the product identifier for reading by the computer vision process. For example, a machine learning algorithm can be used to determine whether one or more of the respective positions within the storage area associated with the missing, unrecognized, or unreadable product identifier contain a unit of the product. This disclosure contemplates that the machine learning algorithm can be executed by the controller 109 in some implementations using traditional vision systems (e.g., pattern recognition), while in other implementations the machine learning algorithm can be executed by the remote system (i.e., offloaded from the inventory management cabinet 2100). Machine learning algorithms can be trained using an existing dataset to perform a specific task such as identify missing, unrecognized, or unreadable product identifiers. Machine learning algorithms are known in the art and therefore not described in further detail below. An example machine learning algorithm is TensorFlow, which is an open source machine learning algorithm known in the art. TensorFlow is only one example machine learning algorithm. This disclosure contemplates using other machine learning algorithms including, but not limited to, neural networks, support vector machines, nearest neighbor algorithms, supervised learning algorithms, unsupervised learning algorithms.

Optionally, in some implementations, the step of inventorying the product based, at least in part, on the information about the product further includes analyzing the images of the product to determine, using a machine learning algorithm, a source of each of the respective units of the product. This is particularly useful when, for example, the product is sourced from multiple vendors or manufacturers. In other words, the inventory management cabinet 2100 can be used to store product from different sources (e.g., contact lenses from different manufacturers). As described above, a computer vision system including an imaging device such as a camera can be used to capture images of both machine readable codes (barcodes, UPC, SKU) and text and graphics, and then imaging processing techniques can be used to decode the product identifiers. This disclosure contemplates that a machine learning algorithm can be used to identify machine readable codes associated with different vendors or manufacturers. This allows the inventory management cabinet 2100 to select the appropriate decoding rules. Alternatively or additionally, a machine learning algorithm can be used to identify the source of a unit of product based on text and/or graphics (even in the absence of machine readable codes). This disclosure contemplates that the machine learning algorithm can be executed by the controller in some implementations, while in other implementations the machine learning algorithm can be executed by the remote system (i.e., offloaded from the inventory management cabinet 2100). Machine learning algorithms can be trained using an existing dataset to perform a specific task such as identify the source of units of the product. Machine learning algorithms are known in the art and therefore not described in further detail below. Example machine learning algorithms are provided above.

Referring now to FIGS. 22A-24C, example inventory management cabinets 2200 according to another implementation are described. The inventory management cabinets of FIGS. 22A-24C use computer vision to inventory product contained in the cabinets as described herein.

FIG. 22A illustrates one aspect where the cabinet 2200 includes a plurality of drawers 2202 (i.e., 4 drawers) and dummy storage area 2204. The cabinet dimensions are 1721.50 mm (67.8 in) height, 600 mm (23.6 in) width, and 650 mm (25.6 in) depth. The dimensions inside the drawer 2202 are 982 mm (38.66 in) width and 545 mm (21.46 in) depth. It should be understood that the configuration (e.g., number of drawers) and dimensions are provided only as examples. This disclosure contemplates providing a cabinet with different configurations and/or dimensions. In FIG. 22A, two imaging devices 2206 are mounted at the top of the cabinet 2200. This disclosure contemplates providing a cabinet with more or less than two imaging devices. The imaging devices 2206 are arranged to capture information about the product located inside the drawers 2202. Optionally, the imaging devices 2206 are mounted flush with the top of the cabinet 2200. For example, each of the imaging devices 2206 is an autofocus digital camera that can capture images of the inside of any one of the four drawers 2202 shown in FIG. 22A. In FIG. 22A, the spacing between the autofocus digital cameras is 491 mm (19.3 in). It should be understood that this spacing is provided only as an example and may have another value. Optionally, the digital cameras do not capture images of the dummy storage area 2204.

FIG. 22B illustrates another aspect where the cabinet 2200 includes a plurality of drawers 2202 (i.e., 4 drawers) without a dummy storage area. The cabinet dimensions are 1413.50 mm (55.6 in) height, 600 mm (23.6 in) width, and 650 mm (25.6 in) depth. The dimensions inside the drawer 2202 are 982 mm (38.66 in) width and 545 mm (21.46 in) depth. It should be understood that the configuration (e.g., number of drawers) and dimensions are provided only as examples. This disclosure contemplates providing a cabinet with different configurations and/or dimensions. In FIG. 22B, three imaging devices 2206 are mounted at the top of the cabinet 2200. This disclosure contemplates providing a cabinet with more or less than three imaging devices. The imaging devices 2206 are arranged to capture information about the product located inside the drawers 2202. Optionally, the imaging devices 2206 are mounted flush with the top of the cabinet 2200. For example, each of the imaging devices 2206 is an autofocus digital camera that can capture images of the inside of any one of the four drawers 2202 shown in FIG. 22B. In FIG. 22B, the spacing between the autofocus digital cameras is 327 mm (12.9 in). It should be understood that this spacing is provided only as an example and may have another value.

As described herein, the images captured by the imaging devices 2206 shown in FIGS. 22A-22B are transmitted to and processed by a controller (e.g., controller 106 of FIGS. 1-12). Optionally, in some implementations, the computer vision methods described herein are triggered by motion sensor(s) (e.g., motion sensor 108 of FIGS. 1-12) and/or product sensors (e.g., presence sensors 110 of FIGS. 1-12). Alternatively or additionally, the cabinets 2200 shown in FIGS. 22A-22B can include one or more visual indicators such as LEDs (e.g., visual indicators 112 of FIGS. 1-12) to illuminate the location of desired product(s) inside the drawers 2202.

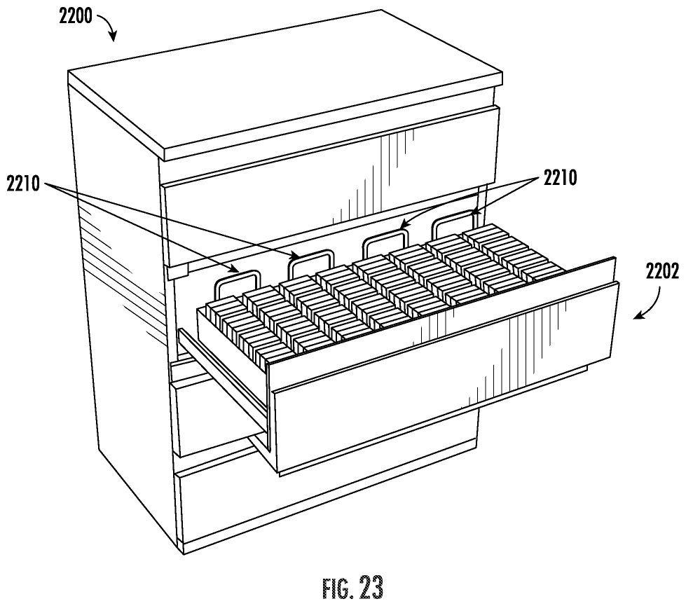

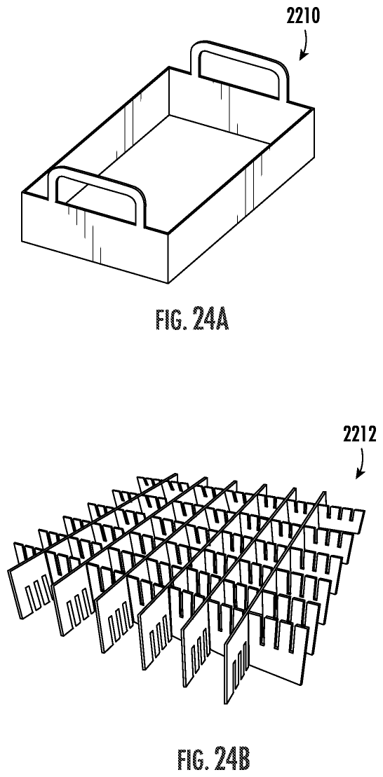

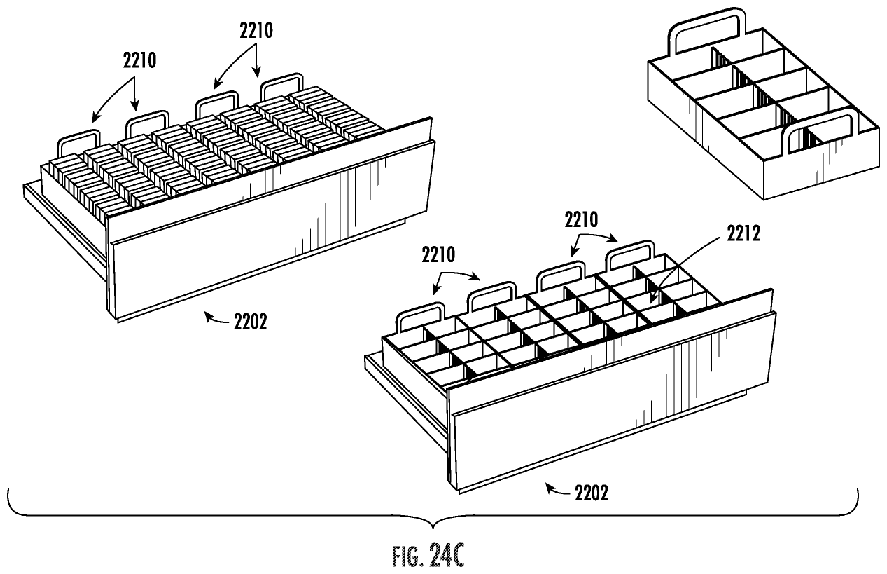

Referring now to FIGS. 23-24C, the cabinet 2200 shown in FIG. 22B with an open drawer 2202 as well as its contents are described. Each drawer 2202 can include one or more trays 2210. It should be understood that the number and/or arrangement of trays 2210 within a drawer is provided only as an example. Additionally, dividers 2212 can be arranged inside the trays 2210. The dividers 2212 create slots for holding units of product. As described herein, the product may be surgical implants including, but not limited to, IOLs or orthopedic implants. The dividers 2212 can be created with different dimensions to accommodate different sized product packaging. Using trays 2210 and dividers 2212, it is possible to provide reconfigurable storage.

Example Computing Device

It should be appreciated that the logical operations described herein with respect to the various figures may be implemented (1) as a sequence of computer implemented acts or program modules (i.e., software) running on a computing device (e.g., the computing device described in FIG. 20), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 20, an example computing device 700 upon which the methods described herein may be implemented is illustrated. It should be understood that the example computing device 700 is only one example of a suitable computing environment upon which the methods described herein may be implemented. Optionally, the computing device 700 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 700 typically includes at least one processing unit 706 and system memory 704. Depending on the exact configuration and type of computing device, system memory 704 may be volatile (such as random access memory (RAM)), non-volatile (such as read-only memory (ROM), flash memory, etc.), or some combination of the two. This most basic configuration is illustrated in FIG. 20 by dashed line 702. The processing unit 706 may be a standard programmable processor that performs arithmetic and logic operations necessary for operation of the computing device 700. The computing device 700 may also include a bus or other communication mechanism for communicating information among various components of the computing device 700.

Computing device 700 may have additional features/functionality. For example, computing device 700 may include additional storage such as removable storage 708 and non-removable storage 710 including, but not limited to, magnetic or optical disks or tapes. Computing device 700 may also contain network connection(s) 716 that allow the device to communicate with other devices. Computing device 700 may also have input device(s) 714 such as a keyboard, mouse, touch screen, etc. Output device(s) 712 such as a display, speakers, printer, etc. may also be included. The additional devices may be connected to the bus in order to facilitate communication of data among the components of the computing device 700. All these devices are well known in the art and need not be discussed at length here.

The processing unit 706 may be configured to execute program code encoded in tangible, computer-readable media. Tangible, computer-readable media refers to any media that is capable of providing data that causes the computing device 700 (i.e., a machine) to operate in a particular fashion. Various computer-readable media may be utilized to provide instructions to the processing unit 706 for execution. Example tangible, computer-readable media may include, but is not limited to, volatile media, non-volatile media, removable media and non-removable media implemented in any method or technology for storage of information such as computer readable instructions, data structures, program modules or other data. System memory 704, removable storage 708, and non-removable storage 710 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 706 may execute program code stored in the system memory 704. For example, the bus may carry data to the system memory 704, from which the processing unit 706 receives and executes instructions. The data received by the system memory 704 may optionally be stored on the removable storage 708 or the non-removable storage 710 before or after execution by the processing unit 706.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

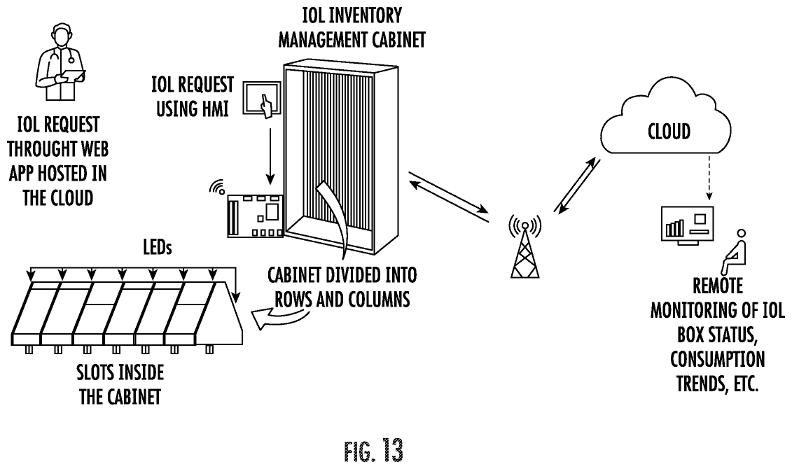

Referring now to FIG. 13, the example smart inventory management cabinet system is comprised of a SMART CABINET and IOL boxes deployed on customer sites, cloud back-end and client applications (web and local interface). The system serves for identification and selection of consigned IOL box inventory and the cabinet control system software reports it to IoT Edge, and it reports the inventory to the cloud in real-time. Events such as IOL box addition or removal by users are tracked by sensors and reconciled with identifying information (e.g., barcode) for proper location and pass on to IoT Edge software to manage/mitigate low inventory scenarios. It uses cameras to read the barcodes based on the commands received from IoT Edge software through IoT Interface.

The SMART CABINET is an IOL box storage box with SMART connectivity. The SMART CABINET contains the high-end single board computer (SBC) as a main processor. Cameras and speakers are connected to SBC. The system interfaces with local user interface, through a graphical user interface (GUI). The example smart inventory management cabinet system is controlled and monitored through IoT Edge device connected with the Cloud. It also has wireless connectivity through Wi-Fi network. The barcode and corresponding slot id will be maintained in local cabinet control system (CCS) software (SW).

The ICD (interface control commands) commands are initiated from IoT Edge and send to cabinet control system through IoT Interface. The CCS sends respective responses to IoT Edge for the commands received from IoT Edge. CCS sends alert message to IoT Edge based on the hardware change events.

For most of the commands received, the CCS SW sends either response/alert message. For any error/vital information, alert messages are sent to IoT edge and the ICD commands received from IoT Edge are sent to CCS SW through IoT Interface. However, for the alert messages sent to IoT Edge, the CCS will not receive any acknowledgement back from IoT Edge. The example smart inventory management cabinet system has the human machine interface (HMI) as user interface. Light emitting diodes (LED) to indicate the slot selection. Cameras are connected through USB port to capture the image and read the IOL box barcode and the LED indication is used to locate the requested IOL box.

When the CCS SW receives the command for full scan, the camera reads the barcode of the IOL boxes and identify the boxes. The image processing techniques are used for the proper identification. The slot id and corresponding barcode details are maintained in CCS local file.

System Inputs

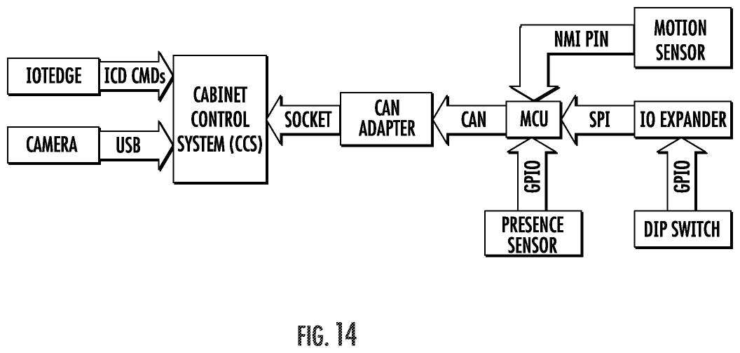

Referring now to FIG. 14, the Cabinet Control System SW is responsible to manage hardware's connected in the cabinet. The CCS SW is connected with below devices as input:

Motion Sensor connected with microcontroller unit (MCU) extension board through general purpose input/output (GPIO). Output of motion sensor is communicated through controller area network (CAN) bus.

Presence Sensor connected with MCU extension board through GPIO. Output of presence sensor is communicated through CAN bus.

Camera connected through universal serial bus (USB) of SBC.

Dual in-line package (DIP) switch is connected in GPIO of IO Expander and MCU gets the switch output through serial peripheral interface (SPI) communication for identifying the MCU ID.

The ICD commands received from IoT Edge are shared to CCS SW through sockets. Internally Command Dispatcher is one of the module in CCS SW and it does the following to execute commands: add the commands into the message queue (first in first out (FIFO) logic); validate received commands; parse the received commands; and send the corresponding events to the respective modules to execute the commands.

The Camera is connected through the USB 3.0 multi ports adapter. When the full scan query is requested from the IoT Edge, CCS invokes the Image Processing Engine module. This Image Processing Engine module does the following to get barcode results: Cameras are triggered to capture the image; Images are captured in parallel with number of cameras for parallel operation shall be preset; Captured images are processed and barcodes are read; and Scanned barcodes are sent to IoT Edge.

Motion sensors are connected to the NMI pin of the MCU extension board. The motion sensor observes the user movement and stores the motion state in queue. When the motion is detected, the following are done: Trigger all the MCU extension board from sleep mode to wakeup mode; Turn on the power of all the presence sensors (IR sensors) connected to MCU extension; MCU extension board sends the motion detected message on the CAN bus.

Presence sensors are connected to the GPIO pin of the MCU extension board. When the user inserts or removes the IOL box, the MCU extension board identifies the slot ID of IOL box insertion or removal based on the presence sensor (IR sensor) input and sends the corresponding message on CAN bus.

System Outputs

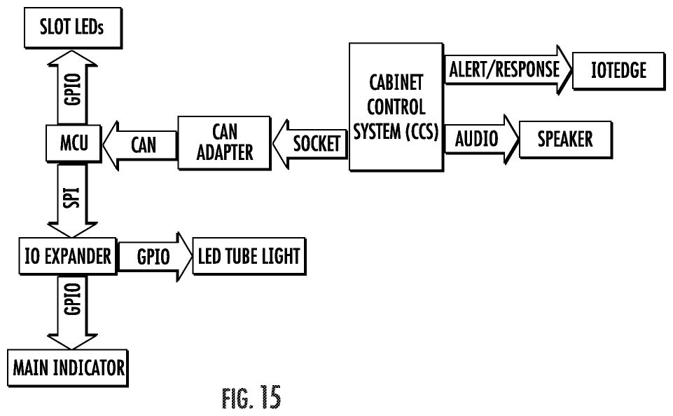

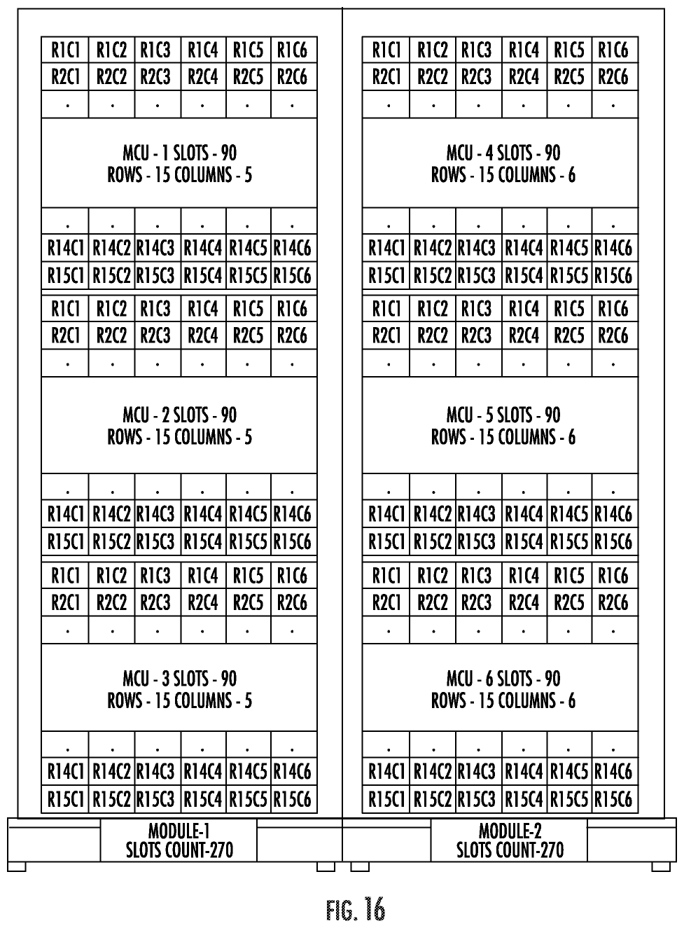

Referring now to FIG. 15, the CCS SW is connected with below devices as output:

LED tube lights are connected in GPIO of IO Expander and controlled by MCU through SPI communication.

Main indicator is connected in GPIO of IO Expander and controlled by MCU through SPI communication.

Slot LEDs are directly connected in GPIO of MCU.

Speaker connected through audio line outputs of SBC.

The CCS SW is directly communicated to MCU extension board through CAN bus. The Main indicator are connected to MCU extension board through SPI communication. The slot LED is LED light which illuminates with corresponding color on slot ID (or group of slots) of requested IOL box to notify user. To achieve this, ICD commands includes the information of slot LED, color information and optional parameters like LED glow time.

The CCS SW plays audio through speaker to give warning about wrong pick up of IOL box. The audio line output of the SBC is connected to the speaker.

LED Tube light is used to get better quality of image when camera starts to capture the images for barcode results of IOL box. This light is turned ON/OFF based on the Scan query start/end events.

Process Model

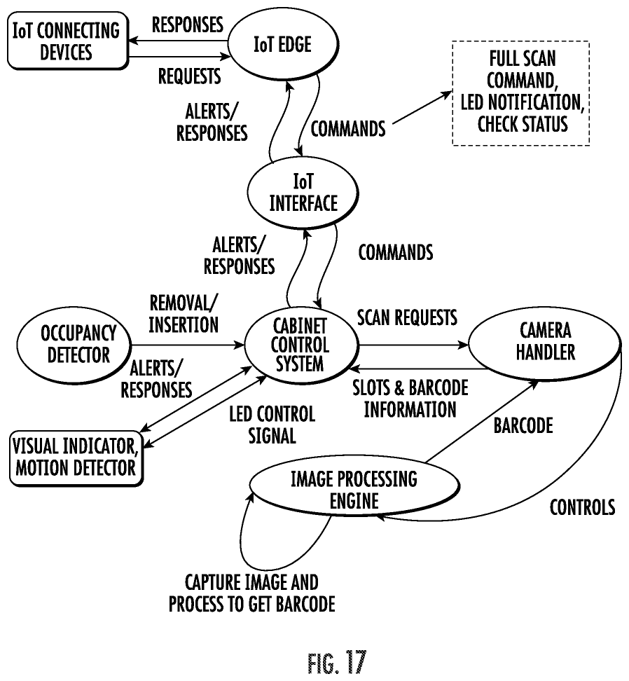

Referring now to FIG. 17, the overall software block diagrams with interfaces between each blocks are shown.

IoT Edge SW is the main interface with user and cloud. All hardware's for IoT connectivity are managed by IoT Edge. The IoT Edge is connected to CCS software through an IoT interface. The IoT interface is using TCP/USB protocol to communicate with IoT Edge.

The ICD (interface control commands) commands are initiated from IoT Edge and send to cabinet system. Responses and alerts are received in response to commands sent and as response to hardware change event.

The overall system level context diagram is shown in the above diagram. For most of the commands received, CCS SW sends response/alert message. For any error/vital information, alert messages are to be sent to IoT edge.

Visual Indicators

The CCS SW is indirectly connected to LED through the CAN bus. The visual notification is used to locate the requested IOL box with slot or group of slots. To achieve this ICD commands includes the information about slot LED, color information and optional parameters.

When the CCS SW receives LED turn ON/OFF commands from IoT Interface, the CCS SW triggers the Visual Indicator to send the turn ON/OFF LED message on CAN bus to MCU extension board to turn on the specified LED with given RGB value through Router application.

Motion Detector

Motion Detector is one of the module in CCS SW. When the motion sensor observes the motion movement and validates the motion change, rises the interrupt in MCU extension board. MCU will send the message on CAN bus indicating that the motion status such as motion started, and motion completed. On the SBC, the router application shall receive the CAN message and decode the message and send the message to Motion Detector. The Motion Detector then sends the message to Post Engine to send it to IoT Edge through IoT Interface.

Presence Detector

Presence Sensor is connected through the GPIO in MCU extension board. Occupancy Detector is one of the module in CCS SW which sends the IR sensor current status query to Router application upon system startup. MCU firmware checks the presence sensor state changes in a polling method. This state change shall happen due to IOL box movement (insertion/removal change). MCU sends the message on CAN bus to indicate the presence sensor status such as Box inserted/retrieved. On the SBC, the router application receives the CAN message decodes it and send the message to Occupancy Detector.

Alarms

The Buzzer Alarm is responsible to turn ON/OFF the speaker based on the event received from Command Dispatcher. It plays audio (unmute before playing) through speaker to give warning about wrong pick up of IOL box. The audio line output of the SBC is connected to the speaker.

### Example 2

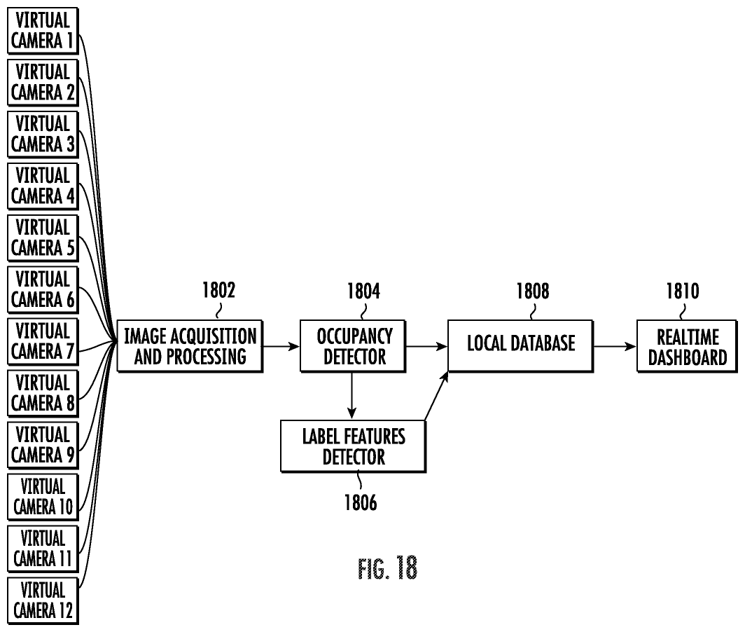

Referring now to FIGS. 18 and 19, an example modular smart inventory management cabinet system integrated with a vision system, cabinet control software, and an IoT interface is illustrated. The integrated system handles translates the physical IOL inventory into inventory data with the use of the vision system, which then the cabinet control system uploads to the cloud for meaningful blocks. The blocks of information are then accessible via the web application.

In FIG. 18, a plurality of cameras 1800 are shown. There are 12 cameras 1800 in FIG. 18. It should be understood that this disclosure contemplates providing a smart inventory management cabinet system with more or less than 12 cameras 1800. At 1802, images captured by the cameras 1800 are timestamped and queued for processing by a trained machine learning (ML) algorithm. At 1804, the system performs occupancy detection for all of the slots in the cabinet, for example, by analyzing captured images for changes from “empty” to “occupied” status or vice versa. Occupancy detection is performed 2 times per second. It should be understood that the rate of 2 times per second is provided only as an example. This disclosure contemplates performing occupancy detection at a higher or lower rate. Images of slots with changes are then cropped and queued for barcode decoding and/or optical character recognition (OCR). At 1806, the system performs barcode decoding and/or OCR to determine the serial number, expiration data, model/variant/diopter, etc. of the product packages. At 1808, information about the occupancy status and product packages obtained at 1804 and 1806 are stored locally by the system, for example, in a database. At 1810, a remote database is updated such that a web-based dashboard can be provided showing a virtual cabinet status including updates in real time.

User then can access the data and use the cabinet features to locate product via pick-to-light technology or identify expired products. Manual product loading or unloading triggers an automated system response to update inventory data.

Occupancy Detector Model

Continuously scans the system every 100 ms for boxes in the corresponding compartments. If a compartment state changes from “empty” to “occupied” this event triggers the Label Features Detector model.

Label Feature Detectors

This model uses either the barcode or OCR to decode the label information. If the barcode cannot be decoded the system will then trigger the OCR. The system will also indicate which box to pick first, if more than one met the locate criteria via blinking the identified lens corresponding LED.

Locate Feature

The user can directly locate a product in a cabinet and the cabinet will display its location in the web app and the product location in the cabinet via pick-to-light.

Expired Product Feature

An expired product locate can be requested by the user. The system will identify all expired products in the cabinet by illuminating the expired product box red via the RGB LED.

Some of the above-described and additional aspects of the invention are further described by the following examples:

Example 1. An inventory management cabinet comprising: a housing defining a storage area, the storage area being configured to receive a product; a plurality of slots arranged within the housing, each of the slots being configured to receive a respective unit of the product; at least one imaging device configured to capture information about the product; and a controller operably coupled to the at least imaging device, the controller comprising a processor and a memory, the memory having computer-executable instructions stored thereon that, when executed by the processor, cause the controller to: detect activity within the storage area of the housing; and control the at least one imaging device to initiate capture of one or more images of the product in response to detecting activity within the storage area of the housing.

Example 2. The inventory management cabinet of example 1, wherein activity within the storage area of the housing is detected using the at least one imaging device.

Example 3. The inventory management cabinet of example 1 or 2, further comprising a motion sensor configured to detect presence of an object within the storage area of the housing.

Example 4. The inventory management cabinet of example 3, wherein activity within the storage area of the housing is detected using the motion sensor.

Example 5. The inventory management cabinet of any one of examples 1-4, further comprising a plurality of presence sensors configured to detect presence of a respective unit of the product in a respective one of the plurality of slots.

Example 6. The inventory management cabinet of example 5, wherein activity within the storage area of the housing is detected using the presence sensors.

Example 7. The inventory management cabinet of example 5 or 6, wherein each of the presence sensors comprises a light emitter and a photodetector.

Example 8. The inventory management cabinet of any one of examples 1-7, wherein the memory has further computer-executable instructions stored thereon that, when executed by the processor, cause the controller to extract information from the one or more images of the product captured by the at least one imaging device.

Example 9. The inventory management cabinet of example 8, wherein extracting information from the one or more images of the product captured by the at least one imaging device comprises:

receiving the one or more images of the product from the at least one imaging device; and

analyzing the one or images of the product to extract respective product identifiers associated with respective units of the product.

Example The inventory management cabinet of example 9, wherein the memory has further computer-executable instructions stored thereon that, when executed by the processor, cause the controller to inventory the product based, at least in part, on the extracted information.

Example 11. The inventory management cabinet of example 10, wherein inventorying the product based, at least in part, on the extracted information comprises: decoding the respective product identifiers associated with the respective units of the product; and using the respective product identifiers, associating the respective units of the product with the respective slots.

Example 12. The inventory management cabinet of any one of examples 9-11, wherein each of the respective product identifiers is a one-dimensional (1D) barcode, a two-dimensional (2D) barcode, a three-dimensional (3D) barcode, a universal product code (UPC), a stock keeping unit (SKU), text, or a graphic.

Example 13. The inventory management cabinet of any one of examples 1-12, further comprising a plurality of imaging devices, each of the imaging devices being configured to capture information about the product located in a respective region of the storage area of the housing.

Example 14. The inventory management cabinet of any one of examples 1-13, further comprising a plurality of visual indicators configured to indicate respective positions of the respective units of the product within the housing.

Example 15. The inventory management cabinet of example 14, wherein the housing comprises an external frame, and wherein at least one of the visual indicators is arranged on or adjacent to the external frame.

Example 16. The inventory management cabinet of example 14, wherein a respective visual indicator is arranged on, within, or adjacent to each one of the respective slots.

Example 17. The inventory management cabinet of any one of examples 14-16, wherein each of the visual indicators is at least one of a light emitter or a graphical display.

Example 18. The inventory management cabinet of any one of examples 1-17, further comprising an output device configured to provide a visual or audible alarm.

Example 19. The inventory management cabinet of example 18, wherein the memory has further computer-executable instructions stored thereon that, when executed by the processor, cause the controller to generate an alarm signal and transmit the alarm signal to the output device.

Example 20. The inventory management cabinet of any one of examples 1-19, further comprising a human machine interface (HMI) configured to provide a communication interface between a user and the inventory management cabinet.

Example 21. The inventory management cabinet of any one of examples 1-20, wherein each of the respective units of the product is a product package.

Example 22. The inventory management cabinet of example 21, wherein the product package includes a surgical implant.

Example 23. The inventory management cabinet of example 22, wherein the surgical implant is an intraocular lens.

Example 24. The inventory management cabinet of example 22, wherein the surgical implant is an orthopedic implant.

Example 25. The inventory management cabinet of example 21, wherein the product package includes a surgical tool.

Example 26. The inventory management cabinet of any one of examples 1-25, wherein the memory has further computer-executable instructions stored thereon that, when executed by the processor, cause the controller to transmit an inventory of the product over a network to a remote system.

Example 27. The inventory management cabinet of example 26, wherein the remote system comprises a database.

Example 28. The inventory management cabinet of any one of examples 1-27, wherein the memory has further computer-executable instructions stored thereon that, when executed by the processor, cause the controller to receive a request for the desired unit of the product.

Example 29. The inventory management cabinet of example 28, wherein the memory has further computer-executable instructions stored thereon that, when executed by the processor, cause the controller to: transmit the request for the desired unit of the product over a network to a remote system; and receive a response from the remote system, the response including a slot where the desired unit of product is located.

Example 30. The inventory management cabinet of example 29, wherein the remote system comprises a database.

Example 31. A modular inventory management system comprising: a first inventory management cabinet according to any one of examples 1-30; a second inventory management cabinet according to any one of example 1-30; and a human machine interface (HMI) configured to provide a communication interface between a user and the first and second inventory management cabinets.

Example 32. An automated method for inventory management comprising:

detecting activity within a storage area of a housing of an inventory management cabinet; automatically initiating capture of one or more images of a product within the storage area of the housing in response to detecting activity within the storage area of the housing; and inventorying the product based, at least in part, using information extracted from the one or more images of the product.

Example 33. The method of example 32, wherein the inventory management cabinet is an inventory management cabinet according any one of examples 1-30.

Example 34. The method of example 32 or 33, further comprising providing the inventory management cabinet according any one of examples 1-30.

Example 35. An inventory management cabinet comprising: a housing defining a storage area, the storage area being configured to receive a product; a plurality of drawers arranged within the housing, each of the drawers comprising a plurality of slots, each of the slots being configured to receive a respective unit of the product; a plurality of imaging device configured to capture information about the product; and a controller operably coupled to the at least imaging device, the controller comprising a processor and a memory, the memory having computer-executable instructions stored thereon that, when executed by the processor, cause the controller to: detect activity within the storage area of the housing; and control the imaging devices to initiate capture of one or more images of the product in response to detecting activity within the storage area of the housing.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. An inventory management cabinet comprising:
a housing defining a storage area, the storage area being configured to receive a product;
a plurality of slots arranged within the housing, each of the slots being configured to receive a respective unit of the product;
at least one imaging device configured to capture information about the product; and
a controller operably coupled to the at least imaging device, the controller comprising a processor and a memory, the memory having computer-executable instructions stored thereon that, when executed by the processor, cause the controller to:
detect activity within the storage area of the housing; and
control the at least one imaging device to initiate capture of one or more images of the product in response to detecting activity within the storage area of the housing.

2. The inventory management cabinet of claim 1, wherein activity within the storage area of the housing is detected using the at least one imaging device.

3. The inventory management cabinet of claim 1, further comprising a motion sensor configured to detect presence of an object within the storage area of the housing.

4. The inventory management cabinet of claim 3, wherein activity within the storage area of the housing is detected using the motion sensor.

5. The inventory management cabinet of claim 1, further comprising a plurality of presence sensors configured to detect presence of a respective unit of the product in a respective one of the plurality of slots.

6. The inventory management cabinet of claim 5, wherein activity within the storage area of the housing is detected using the presence sensors.

7. The inventory management cabinet of claim 5, wherein each of the presence sensors comprises a light emitter and a photodetector.

8. The inventory management cabinet of claim 1, wherein the memory has further computer-executable instructions stored thereon that, when executed by the processor, cause the controller to extract information from the one or more images of the product captured by the at least one imaging device.

9. The inventory management cabinet of claim 8, wherein extracting information from the one or more images of the product captured by the at least one imaging device comprises:
receiving the one or more images of the product from the at least one imaging device; and
analyzing the one or images of the product to extract respective product identifiers associated with respective units of the product.

10. The inventory management cabinet of claim 9, wherein the memory has further computer-executable instructions stored thereon that, when executed by the processor, cause the controller to inventory the product based, at least in part, on the extracted information.

11. The inventory management cabinet of claim 10, wherein inventorying the product based, at least in part, on the extracted information comprises:
decoding the respective product identifiers associated with the respective units of the product; and
using the respective product identifiers, associating the respective units of the product with the respective slots.

12. The inventory management cabinet of claim 9, wherein each of the respective product identifiers is a one-dimensional (1D) barcode, a two-dimensional (2D) barcode, a three-dimensional (3D) barcode, a universal product code (UPC), a stock keeping unit (SKU), text, or a graphic.

13. The inventory management cabinet of claim 1, further comprising a plurality of imaging devices, each of the imaging devices being configured to capture information about the product located in a respective region of the storage area of the housing.

14. The inventory management cabinet of claim 1, further comprising a plurality of visual indicators configured to indicate respective positions of the respective units of the product within the housing.

15. The inventory management cabinet of claim 14, wherein the housing comprises an external frame, and wherein at least one of the visual indicators is arranged on or adjacent to the external frame.

16. The inventory management cabinet of claim 14, wherein a respective visual indicator is arranged on, within, or adjacent to each one of the respective slots.

17. The inventory management cabinet of claim 14, wherein each of the visual indicators is at least one of a light emitter or a graphical display.

18. The inventory management cabinet of claim 1, further comprising an output device configured to provide a visual or audible alarm.

19. The inventory management cabinet of claim 18, wherein the memory has further computer-executable instructions stored thereon that, when executed by the processor, cause the controller to generate an alarm signal and transmit the alarm signal to the output device.

20. The inventory management cabinet of claim 1, further comprising a human machine interface (HMI) configured to provide a communication interface between a user and the inventory management cabinet.

21. The inventory management cabinet of claim 1, wherein each of the respective units of the product is a product package.

22. The inventory management cabinet of claim 21, wherein the product package includes a surgical implant.

23. The inventory management cabinet of claim 22, wherein the surgical implant is an intraocular lens.

24. The inventory management cabinet of claim 22, wherein the surgical implant is an orthopedic implant.

25. The inventory management cabinet of claim 21, wherein the product package includes a surgical tool.

26. The inventory management cabinet of claim 1, wherein the memory has further computer-executable instructions stored thereon that, when executed by the processor, cause the controller to transmit an inventory of the product over a network to a remote system.

27. The inventory management cabinet of claim 26, wherein the remote system comprises a database.

28. The inventory management cabinet of claim 1, wherein the memory has further computer-executable instructions stored thereon that, when executed by the processor, cause the controller to receive a request for the desired unit of the product.

29. The inventory management cabinet of claim 28, wherein the memory has further computer-executable instructions stored thereon that, when executed by the processor, cause the controller to:
transmit the request for the desired unit of the product over a network to a remote system; and
receive a response from the remote system, the response including a slot where the desired unit of product is located.

30. The inventory management cabinet of claim 29, wherein the remote system comprises a database.

31. A modular inventory management system comprising:
a first inventory management cabinet according to claim 1;
a second inventory management cabinet according to claim 1; and
a human machine interface (HMI) configured to provide a communication interface between a user and the first and second inventory management cabinets.

32. An automated method for inventory management comprising:
detecting activity within a storage area of a housing of an inventory management cabinet;
automatically initiating capture of one or more images of a product within the storage area of the housing in response to detecting activity within the storage area of the housing; and
inventorying the product based, at least in part, using information extracted from the one or more images of the product.

33. The method of claim 32, wherein the inventory management cabinet is an inventory management cabinet according to claim 1.

34. The method of claim 32, further comprising providing the inventory management cabinet according to claim 1.

35. An inventory management cabinet comprising:
a housing defining a storage area, the storage area being configured to receive a product;
a plurality of drawers arranged within the housing, each of the drawers comprising a plurality of slots, each of the slots being configured to receive a respective unit of the product;
a plurality of imaging device configured to capture information about the product; and
a controller operably coupled to the at least imaging device, the controller comprising a processor and a memory, the memory having computer-executable instructions stored thereon that, when executed by the processor, cause the controller to:
detect activity within the storage area of the housing; and
control the imaging devices to initiate capture of one or more images of the product in response to detecting activity within the storage area of the housing.
